(19) United States
(12) Patent Application Publication
(10) Pub. No.: US 2004/0030237 A1
(43) Pub. Date: Feb. 12, 2004

(54) Fiducial marker devices and methods

(76) Inventors: David M. Lee, Melbourne Beach, FL (US); Matthew S. Solar, Indialantic, FL (US); Rudy A. Mazzocchi, Indian Harbor Beach, FL (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/454,786

(22) Filed: Jun. 4, 2003

Related U.S. Application Data
(63) Continuation-in-part of application No. 10/206,884, filed on Jul. 29, 2002.
(63) Continuation-in-part of application No. 10/374,677, filed on Feb. 25, 2003.

(51) Int. Cl. A61B 5/05

(52) U.S. Cl. 600/414; 600/426

(57) ABSTRACT

This document discusses, among other things, a fiducial markers capable of use in image guided surgery (IGS) procedures, such as neurosurgery, or other applications. One example includes a positioning instrument with a cap that mates directly to an imageable sphere to perform registration. Another example includes a jointed positioning instrument that, when placed in a base, pivots about a location defined by a center of the imageable sphere when it was in the base. Another example includes a fiducial marker with two imageable spheres defining a line intersecting a desired point on the base. Another example includes a base with a receptacle for receiving a positioning instrument. Another example includes an imageable sphere with a removable imageable portion to allow access to a center of the imageable sphere by a positioning instrument.

### CROSS-REFERENCE TO RELATED APPLICATIONS

This patent application is a continuation-in-part of Mazzocchi et al. U.S. patent application Ser. No. 10/206,884, entitled “FIDUCIAL MARKER DEVICES, TOOLS, AND METHODS”, filed on Jul. 24, 2002, which is assigned to the assignee of the present patent application, and which is incorporated herein by reference in its entirety.

This patent application is a continuation-in-part of Solar et al. U.S. patent application Ser. No. 10/374,677, entitled “FIDUCIAL MARKER DEVICES, TOOLS, AND METHODS,” filed on Feb. 25, 2003, which is assigned to the assignee of the present patent application, and which is incorporated herein by reference in its entirety.

This patent application is also related to Mazzochi et al., U.S. patent application Ser. No. ______, entitled “Fiducial Marker Devices, Tools, and Methods,” filed on even date herewith (Attorney Docket No. 723.063US1), which is assigned to the assignee of the present patent application, and which is incorporated herein by reference in its entirety.

### FIELD OF THE INVENTION

This document relates generally to imaging a patient for performing surgical intervention, and more specifically, but not by way of limitation, to fiducial marker devices and associated tools and methods.

### BACKGROUND

Fiducial markers that can be located and recognized by an imaging system are useful in neurosurgery and other applications. For example, in one technique, multiple fiducial markers are screwed into the patient's skull to define recognizable landmarks that appear on a preoperative image of the patient's brain. Such a bone-anchored fiducial marker typically includes an externally threaded bone-screw portion, which is driven into the skull, and a threaded shaft that rises up and out of the skull from the bone-screw. The threaded shaft typically receives a screwed-on imageable sphere that is visible on a magnetic resonance imaging (MRI) image or computed tomography (CT) image. The multiple fiducial markers on the patient's skull define landmarks on preoperative images that are useful to the physician for planning entry coordinates and a trajectory to a target location in the brain. An image-guided workstation uses these preoperative images and planning to guide the neurosurgeon while actually performing the subsequent surgical procedure.

After the preoperative planning phase, the patient is brought into the operating room so that the planned surgical procedure can be performed. On the operating table, the patient's skull is clamped in a head-frame or otherwise immobilized. In order to use the preoperative images provided by the image-guided workstation to guide the surgeon during the procedure, the patient's skull must first be “registered” to the preoperative images. The registration creates an association between (1) the actual physical location of the fiducial markers on the patient's skull in the operating room and (2) the locations of the images of the fiducial markers visible on the preoperatively-obtained images.

According to one registration technique, a “wand” is used to perform the registration. The wand includes multiple light-emitting diode (LED) locators or reflective locators, which are visible to an infrared or other camera in the operating room. The camera is connected to the image-guided workstation. The locators define the position of the wand in the operating room, including the position of a sharp tip portion of the wand, which is in a known physical relationship to the locators. To register the patient, the imageable spheres are unscrewed from the fiducial marker shafts, and replaced by respective “divots” that are sized and shaped to receive the wand tip. These divots are screwed onto the fiducial marker shafts, such that the maximum depression point of the tip corresponds to the same location as the center of the imageable sphere when the imageable sphere was screwed onto the fiducial marker shaft. A reference divot is also present in the operating room at a known location, such as on the operating table or head-frame. During the patient registration process, the surgeon touches the wand tip to the reference divot, and then to each fiducial marker divot. This permits the image-guided workstation to correlate the actual physical location of the patient's skull to the preoperative images. The physician can then use the wand, in conjunction with the image-guided workstation, to locate an appropriate entry point and trajectory to the target in the brain.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic diagram illustrating generally one example of a fiducial marker and a positioning system including a positioning wand with a semispherical cap.

FIG. 2 is a flow chart illustrating generally one example of using devices such as are shown in the example of FIG. 1.

FIG. 3 is a schematic diagram illustrating generally an alternative example of a positioning wand that includes a ball and a socket or other joint.

FIG. 4 is a flow chart illustrating generally one example of using the devices illustrated in FIG. 3 and FIG. 1.

FIG. 5 is a schematic diagram illustrating generally an alternative example of a locator with two imageable spheres and a base with a built-in registration receptacle.

FIG. 6 is a schematic diagram illustrating generally an alternative example of another locator, with an imageable sphere that includes a removable imageable cone.

FIG. 7 is a schematic diagram illustrating generally a top view of the imageable sphere and included imageable cone of FIG. 6.

FIG. 8 is a schematic diagram illustrating generally an alternative example of a locator including an imageable sphere with a removable imageable cone.

FIG. 9 illustrates a top view of the cone of FIG. 8.

FIG. 10 is a flow chart illustrating generally one example of a method for using the devices illustrated in FIGS. 6-9.

FIG. 11A, FIG. 11B, FIG. 11C, and FIG. 11D are schematic diagrams illustrating generally other examples of locators having imageable spheres that include removable imageable components.

FIG. 12 is a schematic diagram illustrating generally an example of a fiducial marker assembly.

### DETAILED DESCRIPTION

In the following detailed description, reference is made to the accompanying drawings which form a part hereof, and in which is shown by way of illustration specific embodiments in which the invention may be practiced. These embodiments, which are also referred to herein as “examples,” are described in sufficient detail to enable those skilled in the art to practice the invention, and it is to be understood that the embodiments may be combined, or that other embodiments may be utilized and that structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. Furthermore, all publications, patents, and patent documents referred to in this document are incorporated by reference herein in their entirety, as though individually incorporated by reference. In the event of inconsistent usages between this documents and those documents so incorporated by reference, the usage in the incorporated reference(s) should be considered supplementary to that of this document; for irreconcilable inconsistencies, the usage in this document controls.

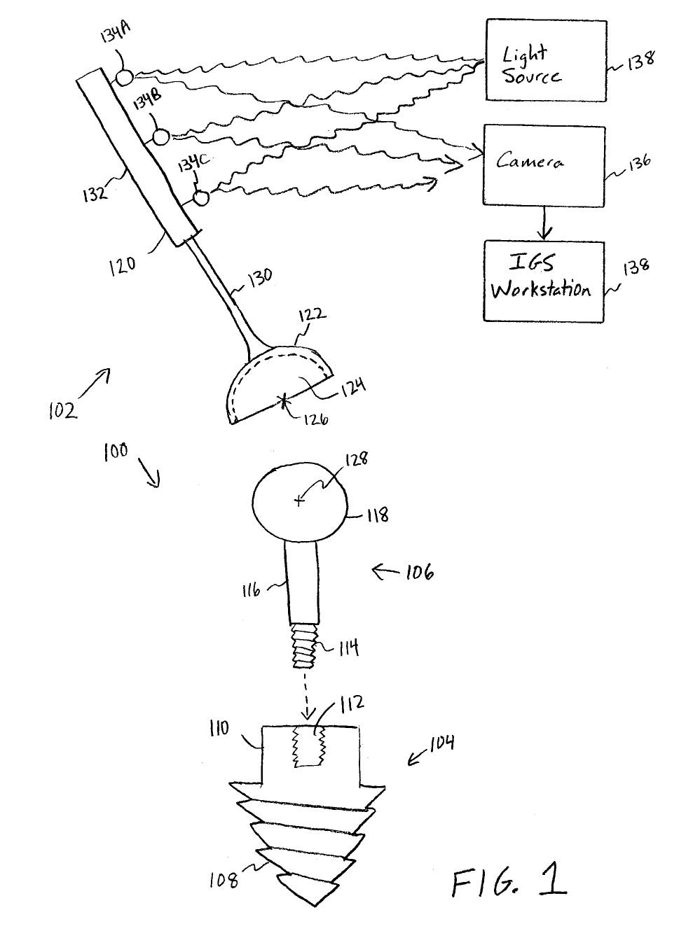

FIG. 1 is a schematic diagram illustrating generally one example of a fiducial marker 100 and a positioning system 102. In this example, the fiducial marker 100 includes an assembly comprising a mounting base 104 and a locator 106. The mounting base 104 includes a self-tapping or other externally threaded distal portion 108. This permits the base 104 to be screwed into a patient's skull or another desired surface. A proximal portion 110 includes a male or female receptacle 112. The receptacle 112 is sized and shaped to receive a complementary male or female receptacle 114 located on a distal portion of the locator 106. In the example of FIG. 1, the receptacle 112 is an internally threaded or other orifice, and the receptacle 114 is an externally threaded or other prong.

In the example of FIG. 1, the locator 106 includes a shaft 116 between the distal receptacle 114 and a proximal imageable sphere 118. The imageable sphere 118 is made from, or contains, a material that provides good contrast on one or more imaging modalities. Examples of suitable imaging systems include, by way of example, but not by way of limitation, magnetic resonance (MR) imaging systems, computed tomography (CT), positron emission tomography (PET), and single photon emission computed tomography (SPECT), X-ray, fluoroscopy, or other radiographic imaging systems, ultrasonic imaging systems, and the like. These imaging modalities permit acquisition of an image of a volume of interest, such as a portion of a subject's brain. The acquired image includes a visible image of the imageable sphere 118, providing a landmark that is located on the subject's skull.

In the example of FIG. 1, the positioning system 102 includes a positioning instrument, which is also sometimes referred to as a positioning wand 120. The wand 120 includes a distal cap 122. The distal cap 122 includes a substantially semispherical orifice 124. The orifice 124 is sized and shaped to fit snugly over the imageable sphere 118 such that a reference point 126 aligns with a center 128 of the imageable sphere 118. The wand 120 includes a shaft 130 between the distal cap 122 and a proximal end 132. The proximal end 132 of the wand 120 includes positioning locators 134A-C that are remotely detectable by a detector portion of the positioning system 102. In this example, the positioning system 102 is implemented as an optical positioning system and the detector is implemented as a camera 136. The positioning locators 134A-C are spherical or other reflectors (or, alternatively, an energy source, such as light-emitting diodes (LEDs)) that are illuminated by a light source 138 for detection by the camera 136. The camera 136 feeds information about the location of the positioning locators 134A-C to an image-guided surgical (IGS) computer workstation 138.

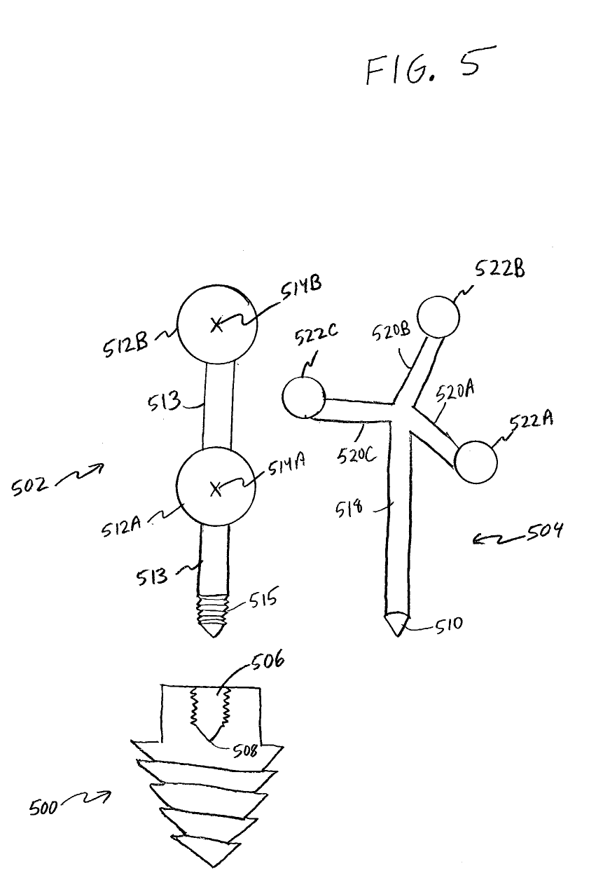

The positioning locators 134A-C are located in a predetermined fixed arrangement with respect to each other and with respect to the reference point 126. Therefore, recognizing the locations of the positioning locators 134A-C using the positioning system 102 allows computation of the location of the reference point 126. Therefore, when the cap 122 is placed upon the sphere 118, this, in turn, permits computation of the location of the center point 128 of the sphere 118. FIG. 1 illustrates the positioning locators 134A-C in a very general conceptual way. One or more of the positioning locators will typically be individually mounted on one or more respective arms extending radially or otherwise from the proximal portion 132 of the wand 120, such as illustrated in FIG. 5.

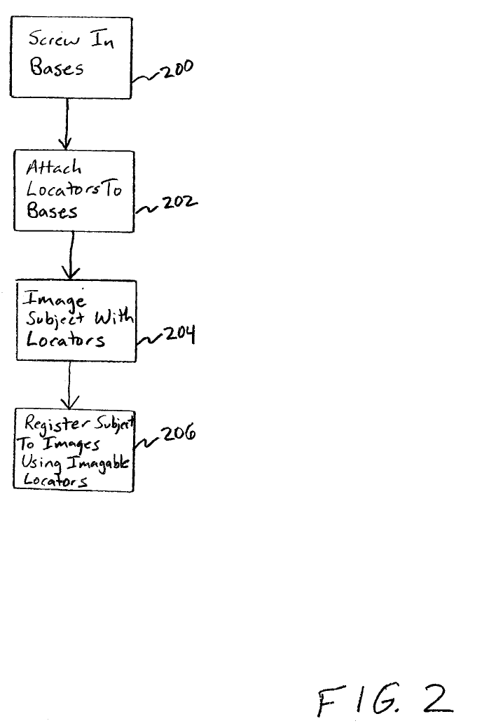

FIG. 2 is a flow chart illustrating generally one example of using devices such as are shown in the example of FIG. 1. In the example of FIG. 2, at 200, several bases (typically at least three or four) are screwed into the subject's skull or other desired surface, such as by using a socket that engages an externally faceted surface such as a hex head of the proximal portion 110 of the base 104. At 202, a locator 106 is attached to each one of the bases 104. At 204, at least one imaging modality is used to take one or more preoperative or other images of the subject's skull, or other desired volume of interest. Images of the locators 106 typically appear with good contrast on the images of the volume of interest. This image information is feed to the IGS workstation 138 for computing the locations, in the three-dimensional space of the images, of the centers 128 of the spheres 118. The subject is then moved to the operating room. At 206, the cap 122 of the wand 120 is placed over each of the spheres 118 to obtain the locations of their centers 128 to register the three dimensional space in which the patient is located to the three dimensional space of the preoperative images. This allows the preoperative images to be used for stereotactically guiding surgical operations on the subject in the operating room. Among other things, the devices shown in FIG. 1 avoid any need for replacing the locators 106 with a golf-tee-like “divot” or “localization cap” for receiving the wand 120. This, in turn, reduces the complexity and cost of the stereotactic procedure.

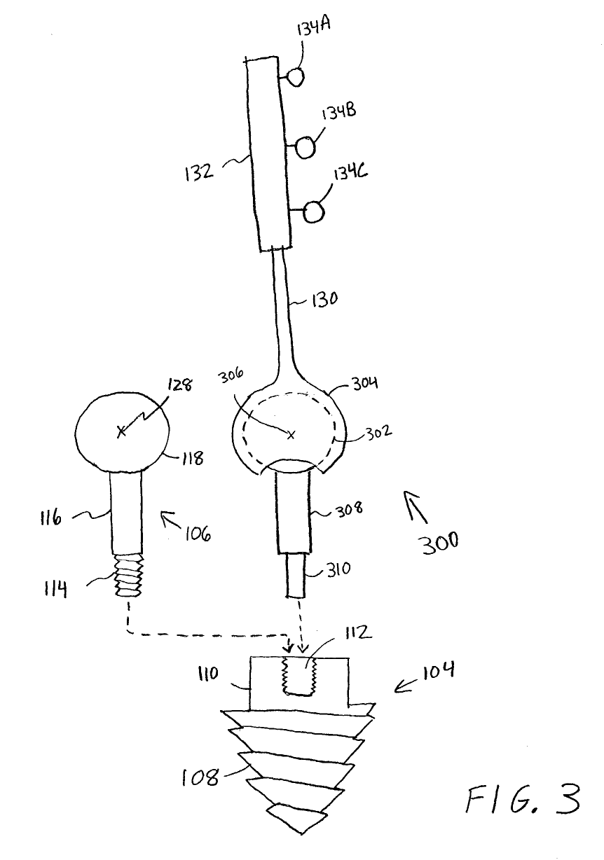

FIG. 3 is a schematic diagram illustrating generally an alternative example of a positioning wand 300, with the base 104 and the locator 106. The positioning wand 300 includes a ball 302 and socket 304 or otherjoint. The ball 302 and socket 304 pivot about a center reference point 306. In this example, the ball 302 is the same size and shape as the sphere 118 of the locator 106. Distal to the ball 302 is a shaft 308 that is the same size as the shaft 116 of the locator 106. Distal to the shaft 116 is a prong (or other male or female receptacle) 310 that is the same length as the prong-like male receptacle 114 of the locator 106. In this example, unlike the externally threaded prong-like receptacle 114 of the locator 106, the prong 310 is not threaded. This permits the prong 310 to be easily inserted into and removed from the receptacle 112 of the base 104.

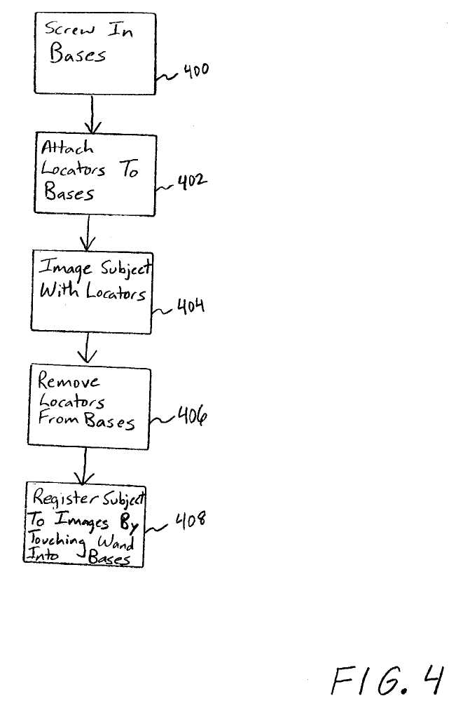

FIG. 4 is a flow chart illustrating generally one example of using the devices illustrated in FIG. 3 and FIG. 1. At 400, the bases 104 are screwed in, such as discussed above. At 402, the locators 106 are attached to respective bases 104, such as discussed above. At 404, the subject is imaged together with the locators 106, such as discussed above. The subject is then moved into the operating room, such as discussed above. At 406, the locators 106 are unscrewed or otherwise removed from the respective bases 104. At 408, the subject is registered to the images. This includes inserting the tip 310 of the positioning wand 300 into the receptacle 112 of each of the respective bases 104. The positioning locators 134 on the wand 300 are in a known relationship to the pivoting center reference point 306, which, in turn, occupies the same location as the center 128 of the sphere 118 when the locator 118 was inserted within the base 104. In this manner, by using the positioning system 102 to determine the locations of the positioning locators 134 on the wand 300, the center point 128 that was occupied by each of the locators 106 can be computed by the IGS workstation 138. Again, among other things, this process avoids any need for replacing the locators 106 with a golf-tee-like “divot” or “localization cap” for receiving the wand 300. This, in turn, reduces the complexity and cost of the stereotactic procedure.

FIG. 5 is a schematic diagram illustrating generally an alternative example of a base 500, a locator 502, and a positioning wand 504. The base 500 is similar, in certain respects, to the base 104. However, in this example, the base 500 includes a receptacle 506 that includes a distal conical “divot” 508, such as for receiving a pointed distal tip 510 of the positioning wand 504. The locator 502 includes two imageable spheres 512A-B. The imageable spheres 512A-B are respectively located on middle and proximal portions of a shaft 513. The spheres 512A-B include respective centers 514A-B that define a line therethrough. When a distal tip 515 of the locator 502 is threaded or otherwise inserted into the receptacle 506 of the base 500, the line through the centers 514A-B extends through the apex (point of maximum depression) of the conical divot 508. In FIG. 5, the positioning wand 504 includes a shaft 518 extending proximally from the distal tip 510 and terminating at or near radial arms 520A-C. The radial arms 520A-C carry respective positioning locators 522A-C.

The method described with respect to FIG. 4 can also be used with the devices shown in FIG. 5. The images of the subject (or other volume of interest) with the locators 502 permit computation of each of the centers 514A-B and of the line defined therebetween. The location of the apex of the divot 508 is located on this line at a known predetermined distance from the centers 514A-B. During registration, at 408, in which the tip 510 of the positioning wand is inserted into the divot 508 of each of the respective bases, the actual locations of the apexes of the divots 508 is computed, because the tip 510 of the positioning wand is in a known spatial relationship to the positioning locators 522. These points of the apexes of the divots 508 are registered to corresponding points in the images that are extrapolated along the line defined by the centers 514 of the imageable spheres 512A-B. Again, among other things, this process avoids any need for replacing the locators 106 with a golf-tee-like “divot” or “localization cap” for receiving the wand 504. This, in turn, reduces the complexity and cost of the stereotactic procedure.

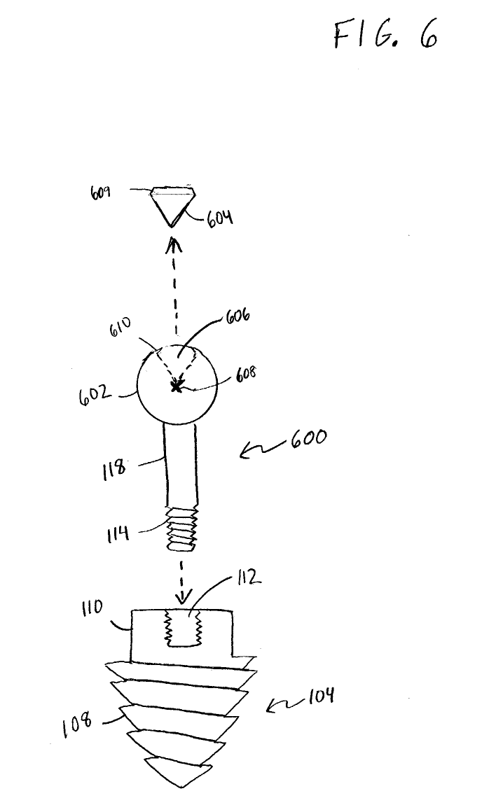

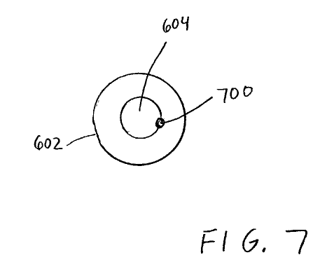

FIG. 6 is a schematic diagram illustrating generally an alternative example of another locator 600 with the base 104. In this example, the locator 600 includes an imageable sphere 602. The imageable sphere 602 includes a removable cone 604 that forms an imageable portion of the imageable sphere 602. Removing the cone 604 creates a conical orifice (also referred to as a divot) 606. The conical orifice 606 has an apex located at a center 608 of the imaging sphere 602. The conical orifice 606 is sized and shaped to permit a tip 510 of a positioning wand 504 to be received therein for performing registration. In one example, the cone 604 snap-fits into the conical orifice 606, such as by a beveled proximal circumferential rim 609 that engages a lip 610 located circumferentially about the proximal base portion of the inverted conical orifice 606, as illustrated in FIG. 1. In one example, the imageable cone 604 and/or the imageable sphere 602 includes a small orifice 700, such as illustrated in the top view of FIG. 7, to facilitate prying the imageable cone 604 out of the imageable sphere 602, such as by using a pick or like instrument to perform this removal.

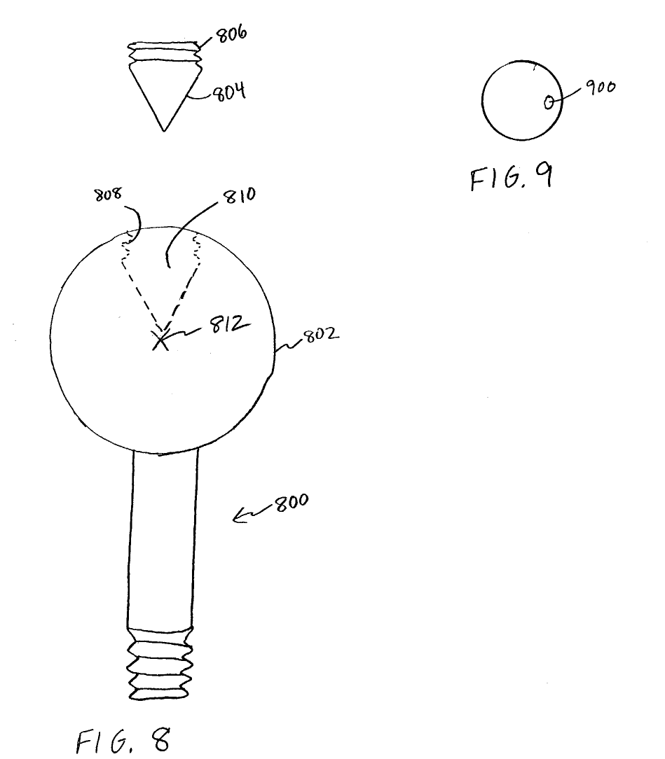

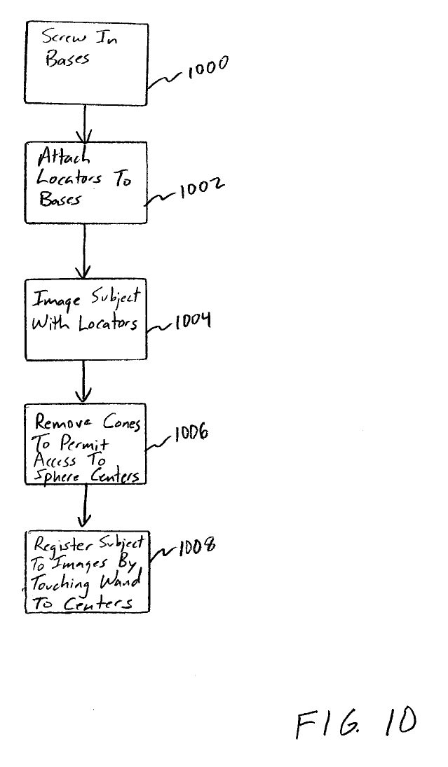

FIG. 8 is a schematic diagram illustrating generally an alternative example of a locator 800 including an imageable sphere 802 with a removable imageable cone 804. In this example, a proximal portion of the cone 804 includes external threads 806 for engaging internal threads 808 of a conical orifice 810 providing a divot for receiving a tip 510 of a positioning wand 504. An apex of the conical orifice 810 corresponds to the center 812 of the imageable sphere 802. FIG. 9 illustrates a top view of the cone 804, including an orifice 900 for receiving a pick or other instrument for unscrewing the cone 804 from the sphere 802 for removing it therefrom.

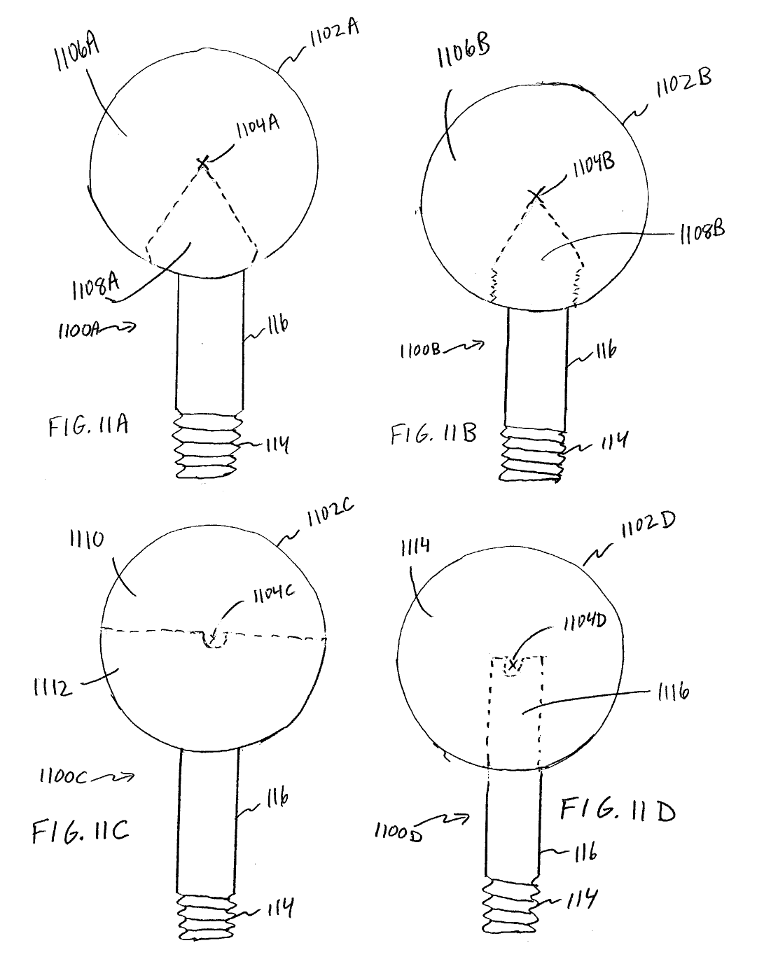

FIG. 10 is a flow chart illustrating generally one example of a method for using the devices illustrated in FIGS. 6-9. In FIG. 10, at 1000, the bases 104 are screwed in, such as discussed above. At 1002, the locators 600 or 800 are attached to respective bases 104, such as discussed above. At 1004, the subject is imaged together with the locators 600 or 800, such as discussed above. The subject is then moved into the operating room, such as discussed above. At 1006, the imageable cones 604 or 804 are pried, unscrewed, or otherwise removed from the respective bases 104. At 1008, the subject is registered to the images. In one example, this includes inserting the tip 510 of the positioning wand 504 into the orifice 610 or 810, such that the tip 510 is located at the center of the imageable sphere 602 or 802. The positioning locators 522 on the wand 504 are in a known relationship to the tip 510 located at the center 608 or 812 of the imageable sphere 602 or 802. In this manner, by using the positioning system 102 to determine the locations of the positioning locators 522 on the wand 504, the center point 608 or 812 can be computed by the IGS workstation 138. Again, among other things, this process avoids any need for replacing the locators 600 or 800 with a golf-tee-like “divot” or “localization cap” for receiving the wand 504. This, in turn, reduces the complexity and cost of the stereotactic procedure. Moreover, accuracy may be enhanced because the tip 510 is located at the actual center 608 or 812 of the imageable sphere 602 or 802, rather than using an intermediate element such as a golf-tee-like “divot” or “localization cap” for receiving the wand 504.

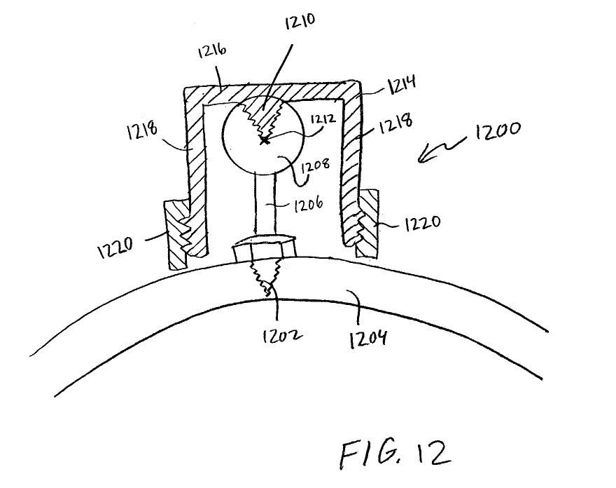

FIGS. 11A-11D are schematic diagrams illustrating generally other examples of locators 1100A-D having imageable spheres 1102A-D that include removable imageable components that allow direct access to the centers 1104A-D of the respective imageable spheres 1102A-D, such as for registration by touching a wand tip 510 thereto. FIG. 1A shows a removable imageable sphere 1106A that is snap-fitted to an imageable inverted cone 1108A on a proximal portion of the shaft 114. FIG. 11B shows a removable imageable sphere 1106B that is threaded onto an imageable inverted cone 1110B on a proximal portion of the shaft 114. The apexes of the inverted cones 1108A and 1108B respectively define the centers 1104A and 1104B of the imageable spheres 1102A and 1102B. FIG. 11C shows a removable imageable hemisphere 1110 that is snap-fitted to a complementary imageable hemisphere 1112 that is attached to a proximal portion of the shaft 114. The snap-fitting provides a small male or female receptacle at the center of the imageable sphere 1102C to which a wand tip can be touched during registration. FIG. 11D shows a removable imageable sphere 1114 that is snap-fitted to an imageable post 1116 extending from a proximal end of the shaft 114. The snap-fitting provides a small male or female receptacle at the center of the imageable sphere 1102C to which a wand tip can be touched during registration. The devices shown in FIGS. 1A-C can be used with the method analogous to that described with respect to the flow chart of FIG. 10.

FIG. 12 is a schematic diagram illustrating generally an example of a fiducial marker assembly 1200. In this example, the fiducial marker assembly 1200 comprises a mounting base 1202, which is attached to a skull 1204, and an imageable fiducial marker locator 1206. The locator 1206 includes an imageable sphere 1208. A removable imageable cone 1210 portion of the imageable sphere 1208 permits access to the center 1212 of the imageable sphere 1208, such as during registration.

In the example of FIG. 12, the cone 1210 is threaded into the other portions of the sphere 1208. The cone 1210 is attached to a protective cap 1214. In the example of FIG. 12, the cap 1214 includes a proximal disk 1216, tangentially extending radially from the removable imageable cone 1210 portion of the imageable sphere 1208. A sleeve 1218 extends from the circumference of the disk 1216 toward the skull 1204. The cap 1214 protects portions of the fiducial marker assembly 1200 from damage. The cap 1214 is either made of a material that is imageable (like the cone 1210 and the other portions of the sphere 1208) or of a different material that is not imageable, i.e., does not provide good contrast on an imaging modality. In a further example, the sleeve 1218 includes external threads that engage internal threads of a cylindrical skirt 1220, which allows the protective cap 1214 to accommodate different scalp thicknesses.

Although the above examples of positioning were illustrated in conjunction with optical positioning systems, certain aspects of such positioning wands can also be used with a wide variety of other remotely detectable positioning systems, such as electric or magnetic field type positioning systems using electric or magnetic positioning locators, articulated arm type positioning systems, etc.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

What is claimed is:

1. An apparatus comprising:
a positioning instrument including a proximal end and a distal end, the distal end including a cap, the cap sized and shaped to mate with an imageable sphere portion of a fiducial marker.

2. The apparatus of claim 1, further comprising at least one remotely detectable positioning locator.

3. The apparatus of claim 2, in which the at least one remotely detectable positioning locator comprises a reflector.

4. The apparatus of claim 2, in which the at least one remotely detectable positioning locator comprises an energy source.

5. A method comprising:
screwing bases into at least one surface of a subject;
attaching imageable spheres to respective ones of the bases;
imaging a volume including the imageable spheres; and
registering a subject to the images, including touching the imageable spheres with a mating positioning instrument.

6. An apparatus comprising:
a positioning instrument, including a proximal end and a distal end, and including a joint between the proximal end and the distal end, in which at least a portion of the distal end is sized and shaped to substantially match an imageable fiducial marker that includes an imageable sphere such that a pivot point of the joint aligns with a position of a center of the imageable sphere when the distal end of the positioning instrument is received within a base from which the imageable fiducial marker has been removed.

7. The apparatus of claim 6, further comprising at least one remotely detectable positioning locator.

8. The apparatus of claim 7, in which the at least one remotely detectable positioning locator comprises a reflector.

9. The apparatus of claim 7, in which the at least one remotely detectable positioning locator comprises an energy source.

10. A method comprising:
screwing bases into at least one surface of a subject;
attaching imageable spheres to respective ones of the bases;
imaging a volume including the imageable spheres;
removing the imageable spheres from the bases; and
registering a subject to the images, including touching the bases with a positioning instrument that pivots about locations that were occupied by centers of the imageable spheres when attached to the respective ones of the bases.

11. An apparatus comprising:
a positioning locator, including a first imageable sphere having a first center and a second imageable sphere having a second center, the first and second centers defining a line therethrough.

12. The apparatus of claim 11, further comprising a base, the base comprising:
a receptacle sized and shaped to receive a portion of the positioning locator, the receptacle including a point that is locatable by a positioning instrument, the point residing along the line defined by the first and second centers when the positioning locator is received by the receptacle.

13. The apparatus of claim 12, in which the receptacle includes an orifice, the orifice comprising:
a threaded proximal portion; and
a conical distal portion.

14. The apparatus of claim 12, further comprising the positioning instrument, the positioning instrument comprising a distal end sized and shaped to be received by the receptacle to locate the point.

15. The apparatus of claim 14, in which the positioning instrument comprises at least one remotely detectable positioning locator.

16. The apparatus of claim 15, in which the at least one remotely detectable positioning locator comprises a reflector.

17. The apparatus of claim 15, in which the at least one remotely detectable positioning locator comprises an energy source.

18. An apparatus comprising:
a base, the base comprising proximal and distal ends, the distal end including external threads, the proximal end including a receptacle sized and shaped to receive a portion of an imageable fiducial marker and also sized and shaped to receive a positioning instrument.

19. The apparatus of claim 18, further comprising an imageable fiducial marker, the imageable fiducial marker including a first imageable sphere having a first center and a second imageable sphere having a second center, the first and second centers defining a line therethrough.

20. The apparatus of claim 19, further comprising a positioning instrument, the positioning instrument comprising a distal end sized and shaped to be received by the receptacle to locate a point on the line defined by the first and second centers when the imageable fiducial marker is received by the receptacle.

21. A method comprising:
screwing bases into at least one surface of a subject;
attaching imageable spheres to respective ones of the bases;
imaging a volume including the imageable spheres;
removing respective portions of the imageable spheres to permit access to centers of the imageable spheres; and
registering a subject to the images, including touching the centers of the imageable spheres with a positioning instrument.

22. An apparatus comprising:
an imageable sphere, the imageable sphere including a removable portion to allow access to a center of the imageable sphere.

23. The apparatus of claim 22, in which the removable portion includes a removable imageable cone.

24. The apparatus of claim 22, in which the removable portion includes a sleeve to at least partially shield the imageable sphere.

25. The apparatus of claim 24, further comprising a skirt coupled to the sleeve.

26. The apparatus of claim 22, further comprising:
a base, the base comprising proximal and distal ends, the distal end including external threads, the proximal end including a receptacle sized and shaped to receive a portion of an imageable fiducial marker that includes the imageable sphere.
